(19) United States
(12) Patent Application Publication
(10) Pub. No.: US 2004/0186449 A1
(43) Pub. Date: Sep. 23, 2004

(54) Liquid-absorbing component for an absorbent article

(76) Inventor: Henri Brisebois, Lachenaie (CA)

Correspondence Address: PHILIP S. JOHNSON, JOHNSON & JOHNSON, ONE JOHNSON & JOHNSON PLAZA, NEW BRUNSWICK, NJ, 08933-7003, US

(21) Appl. No.: 10/393,869

(22) Filed: Mar. 21, 2003

(51) Int. Cl. A61F 13/15; A61F 13/20

(52) U.S. Cl. 604/385.01

(57) ABSTRACT

The present invention provides an absorbent article adapted to be worn in the crotch portion of user's undergarment. The absorbent article comprises a fluid-permeable layer which is oriented toward the user when the absorbent article is in use, a liquid-impervious layer which is oriented toward the user's undergarment when the absorbent article is in use, and at least one liquid-absorbing component located between the fluid-permeable layer and the liquid-impervious layer. The liquid-absorbing component comprises a first side and a second side generally opposite to the first side. The first side includes at least one projection while the second side includes at least one recess.

### FIELD OF THE INVENTION

The present invention relates to a disposable absorbent article such as a sanitary napkin. More specifically, the present invention relates to a disposable absorbent article featuring one or more liquid-absorbing components having a configuration which reduces raw material expenditure during its manufacture.

### BACKGROUND OF THE INVENTION

The term “sanitary napkin”, as used herein, refers to an absorbent article that is worn by females in their undergarments adjacent to the pudendal region, and which is intended to absorb and contain the various exudates that are discharged from the body (e.g., blood, menses, vaginal discharges and urine). Hence, the term “sanitary napkin” encompasses pantiliners in addition to catamenial devices. The term “disposable” refers to articles that are intended to be discarded after a single use and preferably recycled, composted, or otherwise disposed of in an environmentally friendly manner. (That is, they are not intended to be laundered or otherwise restored or reused as an absorbent article.)

Disposable sanitary napkins are articles which are produced in mass. From the standpoint of the manufacturer, it is therefore important to reduce the amount of material required to make the sanitary napkins such as to lower production costs. This is not a trivial task since a reduction in the amount of material in one or more components of the sanitary napkin will usually decrease its performance in use.

Accordingly, an objective of the present invention is to provide a liquid-absorbing component that is suitable for use in a disposable absorbent article's absorbent system, and which is made with a lesser amount of material than traditional liquid-absorbing components. Despite being made with a lesser amount of material than traditional liquid-absorbing components, the liquid-absorbing component according to the present invention does not significantly increase the absorbent article's risk of failure.

### SUMMARY OF THE INVENTION

The present invention provides an absorbent article adapted to be worn in the crotch portion of user's undergarment. The absorbent article comprises a fluid-permeable layer which is oriented toward the user when the absorbent article is in use, a liquid-impervious layer which is oriented toward the user's undergarment when the absorbent article is in use, and at least one liquid-absorbing component located between the fluid-permeable layer and the liquid-impervious layer. The liquid-absorbing component comprises a first side and a second side generally opposite to the first side. The first side includes at least one projection while the second side includes at least one recess.

The first side of the liquid-absorbing absorbing component generally includes a first contour and the second side includes a second contour. In one embodiment of the invention, the first contour and the second contour are capable of substantial coincident alignment when the first side is subject to an imaginary translation along a longitudinally extending centerline of the liquid-absorbing component.

In another aspect of the invention, a method of making a liquid-absorbing component for an absorbent article comprises providing a continuous web of liquid-absorbing material and separating a portion of the liquid-absorbing material from the continuous web to form a liquid-absorbing element. The liquid-absorbing element includes a first side and a second side generally opposite the first side. The first side includes at least one projection and at least one recess.

### BRIEF DESCRIPTION OF THE DRAWINGS

A detailed description of preferred embodiments of the present invention is provided herein below with reference to the following drawings, in which:

FIG. 1 is a top plan view of an absorbent article having a liquid-absorbing component according to a non-limiting example of implementation of the invention, the absorbent article's fluid-permeable cover layer being shown as a see-through layer in order to illustrate the configurations of the inner layers;

FIG. 2 is a cross-sectional view taken along line 2-2 in FIG. 1;

FIG. 3 is a top plan view of the second liquid-absorbing component shown in FIG. 1;

FIG. 4 is a top plan view of the first liquid-absorbing component shown in FIG. 1;

FIG. 5 is a top perspective view of a continuous strip of absorbent material in roll form and a depiction of the second liquid-absorbing component of FIG. 3 being made therefrom;

FIG. 6 is a top plan view of a sheet-like absorbent element which has been die-cut from a continuous web of absorbent material;

FIG. 7 is a top plan view of a multi-layer absorbent component formed by folding the sheet-like absorbent element shown in FIG. 6; and

FIG. 8 is a side view of the multi-layer absorbent component of FIG. 7, illustrating how the sheet-like absorbent element of FIG. 6 is folded to form the multi-layer absorbent component of FIG. 7.

In the drawings, embodiments of the invention are illustrated by way of example. It is to be expressly understood that the description and drawings are only for purposes of illustration and as an aid to understanding, and are not intended to be a definition of the limits of the invention. Throughout the drawings, identical components are designated by the same reference numerals.

### DETAILED DESCRIPTION

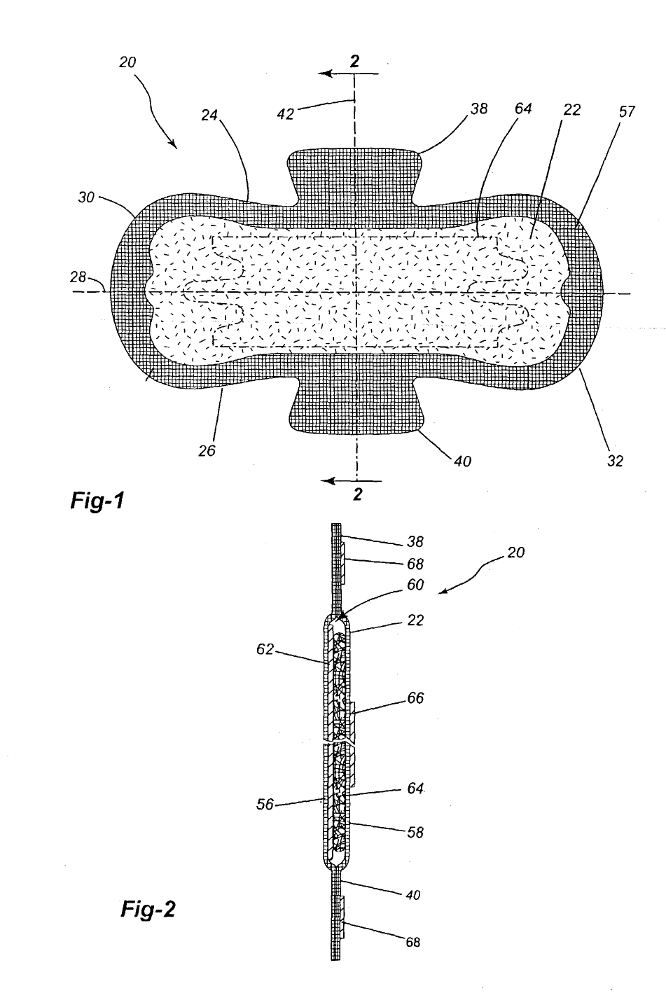

With reference to FIGS. 1 to 2, there is shown a disposable absorbent article 20 such as, for example, a panty liner or a sanitary napkin. The absorbent article 20 generally includes a main body 22 having two mutually opposing longitudinally extending sides 24, 26, and an imaginary longitudinally extending centerline 28 running down the center of the absorbent article 20, and generally equidistant from the longitudinally extending sides 24, 26. Moreover, the main body 22 also has two generally opposing transversely extending sides 30, 32, and an imaginary transversely extending centerline 42 that runs across the body of absorbent article 20, thereby intersecting the imaginary longitudinally extending centerline 28.

The term “longitudinal” or “longitudinally extending,” as used herein, refers to a line, axis or direction in the plane of the absorbent article 20 that is generally aligned with (e.g., approximately parallel to) a vertical plane which bisects a standing wearer into left and right body halves when the absorbent article 20 is worn. The term “transverse” or “transversely extending” as used herein, refers to a line, axis or direction in the plane of the absorbent article 20 that is generally aligned with (e.g., approximately parallel to) a vertical plane which bisects a standing wearer into rear and front body halves when the absorbent article 20 is worn.

Under a specific non-limiting example of implementation, the main body 22 of the absorbent article 20 has a longitudinal shape. When the main body 22 is of such a configuration, the transversely extending sides 30, 32 are the shorter opposing sides while the longitudinally extending sides 24,26 are the longer opposing sides. It should be understood, however, that main bodies 22 of varying shapes remain within the scope of the present invention.

In the example of implementation depicted in FIG. 1, the longitudinally extending sides 24, 26 define flaps 38 and 40 respectively that are adjoined to the main body 22. Each flap 38, 40 is preferably shaped as an isosceles trapezoid, although alternative shapes are contemplated. It should be expressly noted, however, that flaps are not an essential component of the present invention, as an absorbent article without flaps and embodying the present inventive principle can be realized.

FIG. 2 shows that absorbent article 20 has a laminate structure. More specifically, main body 22 includes: a fluid-permeable cover layer 56, which will face the body of a user when the absorbent article 20 is in use; a liquid-impervious barrier layer 58, which will face the environment (i.e. away from the body of the user, and in almost all cases the user's undergarment) when the absorbent article 20 is in use; and an absorbent system 60 therebetween. The absorbent system 60 can include one or more liquid-absorbing components. In the example of implementation depicted in FIGS. 1 to 2, the absorbent system 60 has two liquid-absorbing components, namely a first liquid-absorbing component 62 that includes a single layer of material (commonly known as “transfer layer”) and a second liquid-absorbing component 64 (commonly known as “absorbent core”) that also includes a single layer of material. It should be noted, however, that the number of layers of material which form the first and the second liquid-absorbing components 62 and 64 is not essential, and that the liquid-absorbing components 62 and 64 may include more than one layer of material.

It should also be expressly noted that the first liquid-absorbing component 62 is optional and that the absorbent system 60 may include only the second liquid-absorbing component 64.

The fluid-permeable cover layer 56 and the liquid-impervious barrier layer 58 are sealed together along their respective peripheral edges to form a peripheral flange seal 57. Peripheral flange seal 57 extends continuously around the absorbent system 60 to completely enclose the same. A variety of flange seal configurations is within the scope of this invention.

Each of these layers of the absorbent article 20 will now be described in further detail below.

Fluid-Permeable Cover Layer

The fluid-permeable cover layer 56 is the top layer of the absorbent article 20. The purpose of the fluid-permeable cover layer 56 is to provide an interface that would normally contact the body of the user when the absorbent article 20 is in use. The fluid-permeable cover layer 56 is porous to liquids since its main function is to capture as quickly as possible a discharge of bodily exudate and transfer it to the absorbent system 60 underneath.

Under one specific example of implementation, the fluid-permeable cover layer 56 is formed from an apertured thermoplastic film. Such films are common in the art. Because of the high porosity of such films, they accomplish the function of quickly transferring body exudate to the inner layers (i.e. the absorbent system 60) of the absorbent article 20.

The fluid-permeable cover layer 56 can also be made of fibrous materials, such as non-woven fibrous materials. The fluid-permeable cover layer 56 may be composed of only one type of fiber, such as polyester, polypropylene, polyethylene, or may be composed of bicomponent or conjugate fibers having a low melting point component and a high melting point component. Bicomponent fibers may include, for example, a polyester core and a polyethylene sheath. The use of appropriate bicomponent materials results in a fusible non-woven fabric. Using a fusible fabric increases the ease with which the fluid-permeable cover layer 56 may be mounted to the liquid-impervious barrier layer 58 at the flange seal 57.

The fibers may be selected from a variety of natural and synthetic materials such as nylon, polyester, rayon (in combination with other fibers), cotton acrylic fiber and the like and combinations thereof. It will be evident to the person skilled in the art that a wide variety of other types of non-woven fabric materials can also be used.

Absorbent System—First Liquid-Absorbing Component

Adjacent to the fluid-permeable cover layer 56 on its inner side and bonded thereto is the first liquid-absorbing component 62 that forms part of the absorbent system 60. The first liquid-absorbing component 62 provides the means of receiving body fluid from the fluid-permeable cover layer 56 and holding it until the underlying second liquid-absorbing component 64 has an opportunity to absorb the fluid.

The first liquid-absorbing component 62 may be made from one or more layers of material. The example of implementation of the invention shown in the drawings illustrates a form of realization including a single layer of material.

The first liquid-absorbing component 62 is, preferably, more dense than and has a larger proportion of smaller pores than the fluid-permeable cover layer 56. These attributes allow the first liquid-absorbing component 62 to contain body fluid and hold it away from the outer side of the fluid-permeable cover layer 56, thereby helping to prevent the fluid from re-wetting the fluid-permeable cover layer 56 and its surface. However, the first liquid-absorbing component 62 is, preferably, not so dense as to prevent the passage of the fluid into the underlying second liquid-absorbing component 64.

The first liquid-absorbing component 62 may be composed of fibrous materials, such as wood pulp, polyester, rayon, flexible foam, or the like, or combinations thereof. The first liquid-absorbing component 62 may also comprise thermoplastic fibers for the purpose of stabilizing the layer and maintaining its structural integrity. The first liquid-absorbing component 62 may be treated with surfactant on one or both sides in order to increase its wettability, although generally it is relatively hydrophilic and may not require treatment. The first liquid-absorbing component 62 is preferably bonded on both sides to the adjacent layers, i.e. the fluid-permeable cover layer 56 and the underlying second liquid-absorbing component 64.

Absorbent System—Second Liquid-Absorbing Component

Immediately adjacent to and bonded to the first liquid-absorbing component 62 is the second liquid-absorbing component 64.

As is the case with the first liquid-absorbing component 62, the second liquid-absorbing component 64 may also be made from one or more layers of material. FIGS. 1 to 2 illustrate a form of realization including a single layer of material.

The second liquid-absorbing component 64 has a large liquid-holding capacity and it is extremely retentive. Second liquid-absorbing component 64 may comprise pulp fluff material and may optionally include other absorbent materials or non-absorbent materials such as conjugate fibers, fusible fibers, binders, sphagnum moss, superabsorbents, and the like and combinations thereof.

In one specific form of realization, the second liquid-absorbing component 64 is a blend or mixture of cellulosic fibers and superabsorbent polymers (SAP) disposed in and amongst fibers of that pulp. For the purposes of the present invention, the term “superabsorbent polymer” (or “SAP”) refers to materials that are capable of absorbing and retaining at least about 10 times their weight in body fluids under a 0.5 psi pressure. The SAP particles may be inorganic or organic crosslinked hydrophilic polymers, such as polyvinyl alcohols, polyethylene oxides, crosslinked starches, guar gum, xanthan gum, and the like. The particles may be in the form of a powder, grains, granules, or fibers. Continuing with the form of realization mentioned above, the second liquid-absorbing component 64 is a material containing from about 40 weight percent to about 95 weight percent cellulosic fibers; and from about 5 weight percent to about 60 weight percent SAP.

The material generally has a water content of less than about 10 weight percent. As used herein, the phrase “weight percent” means weight of substance per weight of final material. By way of example, 10 weight percent SAP means 10 g/m² SAP per 100 g/m² basis weight of the material.

Cellulosic fibers that can be used in the second liquid-absorbing component 64 are well known in the art and include wood pulp, cotton, flax and peat moss. However, wood pulp is preferred. Moreover, although both softwood and hardwood pulp species are useful, softwood pulp species are preferred. It is not necessary to treat cellulosic fibers with chemical debonding agents, cross-linking agents and the like for use in the present material.

Main Body—Liquid-impervious Barrier Layer

Underlying the absorbent system 60 is the liquid-impervious barrier layer 58 which comprises liquid-impervious film material in order to prevent liquid that is entrapped in the absorbent system 60 from egressing the absorbent article 20 and staining the wearer's undergarment. The liquid-impervious barrier layer 58 can be made of polymeric film, such as polyethylene or a polyethylene/ethylvinyl acetate (EVA), which are both inexpensive and readily available. The polymeric film is capable of fully blocking the passage of liquid or gas that may emanate from the absorbent system 60. In a variant, breathable films may be used that allow passage of moisture while blocking liquid.

Adhesives

To secure the absorbent article 20 to the undergarment of a wearer, the liquid-impervious barrier layer 58 is provided with an area of standard adhesive material 66 (“adhesive”) on the environmental (i.e. undergarment) facing surface thereof. Additionally, there is an adhesive 68 located on each of the flaps 38, 40. A single standard release paper (not shown) covers the adhesive 66 positioned on the underside of the main body 22 while the adhesive 68 on the flaps 38, 40 are covered by respective release papers (not shown). The release papers are of conventional construction (silicone coated wet-laid Kraft wood pulp).

Configurations of the Liquid-Absorbing Components

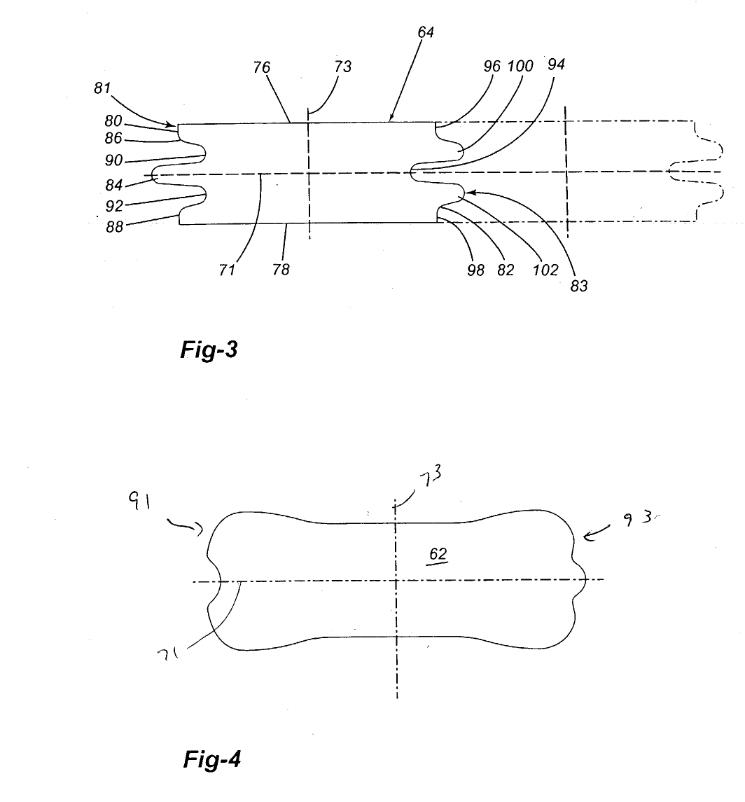

FIG. 3 shows a non-limiting example of implementation of the second liquid-absorbing component 64 in accordance with the present invention. As shown, the second liquid-absorbing component 64, which generally includes a single layer of absorbent material, features a generally longitudinal shape and includes a longitudinally extending centerline 71 as well as a transversely extending centerline 73. Moreover, second liquid-absorbing component 64 also comprises a pair of opposite generally straight longitudinally extending sides 76, 78 and a pair of opposite transversely extending sides 80, 82. The transversely extending sides 80, 82 are respectively characterized by contours 81, 83 which are capable of at least partially engaging one another when subjected to an imaginary translation along the longitudinally extending centerline 71. In one embodiment, the contours 81, 83 are capable of substantially coincident alignment with one another when transversely extending side 81 is subject to an imaginary translation along the longitudinally extending centerline 71. In other words, if contour 81 is subject to an imaginary translation along the longitudinally extending centerline 71 towards contour 83, the contours 81,83 are capable of occupying essentially the same points in space.

In particular, the contour 81 of the transversely extending side 80 has a central projection 84, two lateral projections 86 and 88, and two recesses 90 and 92 between the projections 84, 86 and 84, 88 respectively. In contrast, the contour 83 of the transversely extending side 82 has recesses 94, 96 and 98 capable of matingly receiving the projections 84, 86 and 88 respectively. Furthermore, the contour 83 has projections 100,102 capable of matingly engaging the recesses 90 and 92 of contour 81.

Thus, if transversely extending side 80 was progressively translated in an imaginary fashion along the longitudinally extending centerline 71 up to transversely extending side 82 (this is illustrated via the second liquid-absorbing component shown in dashed lines in FIG. 3), the former would engage at least a portion of the latter such that corresponding projections and recesses on both transversely extending sides would at least partially penetrate one another. Preferably, as shown in FIG. 3, the projections would matingly engage the recesses. By “mating engagement”, it is meant that the outline of the projection corresponds to the outline of the recess such that when the projection and the recess are engaged, the apex and sides of the projection will engage the bottom and sides of the recess, respectively, without any substantial gaps remaining therebetween.

One of the advantages of the above arrangement, among others, is a reduction in the amount of raw material in the area of the transversely extending sides 80, 82. Such selective raw material reduction makes the second liquid-absorbing component 64 more economical to produce without significantly increasing the risk of product failure. In use, body exudate is usually deposited at a point of the absorbent article 20 that corresponds to the center of the second liquid-absorbing component 64 (i.e., where longitudinally extending centerline 71 and transversely extending centerline 73 intersect). Accordingly, the degree of utilization of the absorption capacity is maximal at the point of fluid impact and progressively diminishes toward the transversely extending sides 80, 82. For heavy flows, some absorption capacity may still be required at the very edge of the transversely extending sides 80, 82. However, this required absorption capacity is limited. In short, the selective raw material reduction accomplishes two objectives, one being to maintain an adequate level of absorption capacity at a certain distance from the center of the second liquid-absorbing component 64 and at the same time reducing the total amount of raw material required to produce the latter.

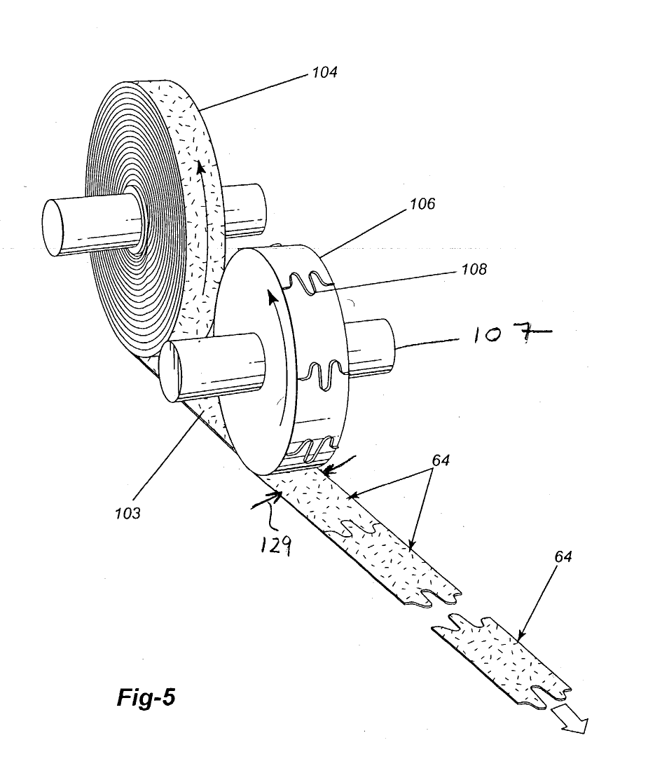

FIG. 5 schematically illustrates an exemplary process for producing discrete second liquid-absorbing components 64 as depicted in FIG. 3. As shown, a roll 104 of a continuous strip of liquid-absorbing material 103 is progressively unwound and processed at a die-cutting station 106 including a roller 107 with a circumferential face having a plurality of cutting blades 108. The cutting blades 108 are patterned according to the transversely extending sides 80, 82 depicted in FIG. 3. Accordingly, when cutting blade 108 severs the strip of liquid-absorbing material 103, it simultaneously creates the transversely extending side of one liquid-absorbing component as well as the transversely extending side of another liquid-absorbing component.

Note that the strip of liquid-absorbing material 103, as shown in the drawings, is a single layer. Alternatively, the strip of liquid-absorbing material 103 may be formed as a combination of strips of material, such as two strips one on top of the other forming one compound strip, or one or more strips partially overlapping one another.

It should be expressly noted that numerous variations in the specific contours 81, 83 of the transversely extending sides 80, 82 could be envisioned without departing from the spirit of the invention. In addition, the same contour arrangement can be applied to any other liquid-absorbing component of the absorbent system 60. FIG. 4, for example, depicts a first liquid-absorbing component 62 having transversely extending sides which differ from those of the second liquid-absorbing component 64, but which are nonetheless capable of matingly engaging one another.

Although the above figures all depict embodiments in which it is the transversely extending sides of the liquid-absorbing components that are capable of at least partially engaging one another, it should be expressly understood that liquid-absorbing components having longitudinally extending sides such as the longitudinally extending sides 76 in FIG. 3 that are capable of at least partially engaging one another also remain within the present inventive concept.

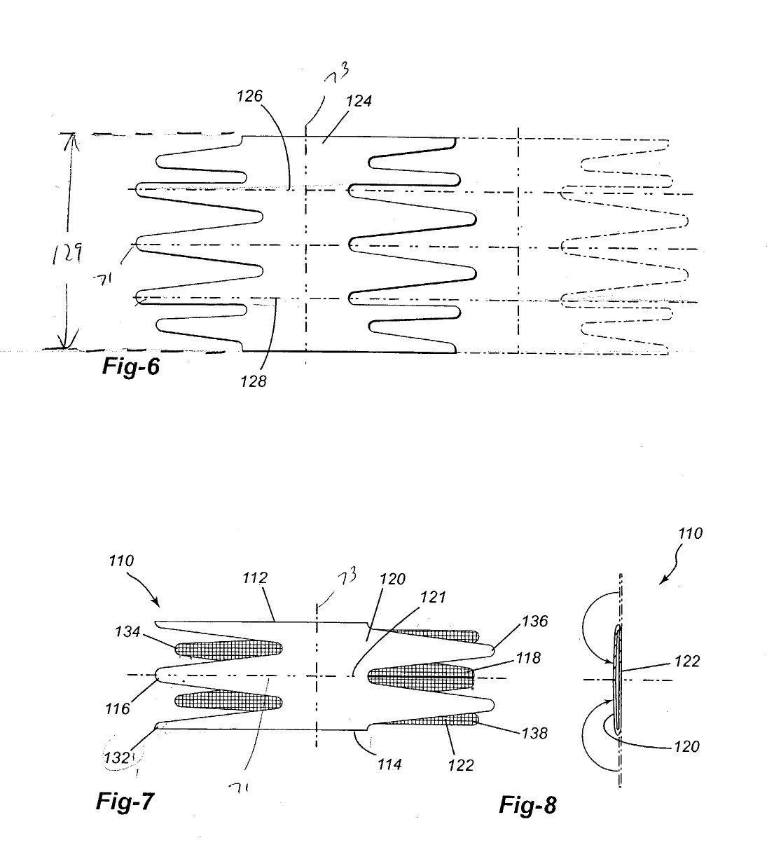

FIGS. 6 to 8 illustrate a non-limiting example of a multi-layer absorbent component 110 for use with the absorbent system 60 shown in FIG. 1. The multi-layer absorbent component 110 can be used as either the first liquid-absorbing component 62, as the second liquid-absorbing component 64, or as both.

FIG. 7 shows a sheet-like absorbent element 124 folded along fold lines 126, 128 to form an absorbent component 110. The absorbent component 110 is therefore in a folded state ready for use in the absorbent article 20. The multi-layer absorbent component 110 has a longitudinal shape with longitudinally extending sides 112 and 114 and transversely extending sides 116, 118. The absorbent component 110 has two superposed, non-coextensive layers, namely an upper layer 120 and a lower layer 122 that are formed by, for example, folding component 110 as described above (Note that the lower layer 122 is marked with cross-hatchings in FIG. 7.). By “non-coextensive”, it is meant that the upper layer 120 and the lower layer 122 have transversely extending sides with outlines which do not substantially overlie one another when they are in a folded state. The transversely extending side 116 includes a first contour 132 defined by the upper layer 120 and a second contour 134 defined by the lower layer 122. Similarly, the transversely extending side 118 has a first contour 136 defined by the upper layer 120 and second contour 138 defined by the lower layer 122. The contours are patterned such that when one of the contours of the transversely extending sides 116 is translated along the longitudinally extending centerline 121, it will at least partially engage the contour from the other transversely extending side 118. In the specific example shown in FIGS. 6-8, the first contour 132 of the transversely extending side 116 matingly engages the first contour 136 of the transversely extending side 118. Similarly, the second contour 134 of the transversely extending side 116 matingly engages the second contour 138 of the transversely extending side 118. Each pair of matingly engaging contours includes at least one projection penetrating a conforming recess.

The multi-layer absorbent component 110 is manufactured from the single sheet-like absorbent element 124 that is then C-folded as shown in FIG. 8 about fold lines 126 and 128. The fold lines 126 and 128 thus form the longitudinally extending sides 112 and 114 and serve to attach an upper layer to a lower layer in the multi-layer absorbent component. Other fold patterns are possible without departing from the spirit of the invention.

Although not shown, a method of making the multi-layer absorbent component 110 could include providing a continuous strip of liquid-absorbing material in roll form, and progressively unwinding it and processing it at a die-cutting station including a roller with a circumferential face having a plurality of cutting blades. The cutting blades are patterned according to the contour of the transversely extending sides of the sheet-like elements 124. Accordingly, when a cutting blade severs the strip of absorbent material, it simultaneously creates one transversely extending side of one sheet-like element 124 and the transversely extending side of another sheet-like element 124. The sheet-like elements 124 are then processed by a processing station of a type known in the art that C-folds the sheet-like elements 124 to form individual multi-layer absorbent components 110.

Since the absorbent structures shown in FIGS. 6 to 8 are made by folding a single layer of material, the width 129 of the strip, while in roll form, is wider than the transverse dimension of the final multi-layer absorbent component. This larger width 129 allows the roll to be built up to a larger diameter than would be the case when the strip is of the same width as the absorbent component (as in FIG. 5). A larger width creates a more stable roll, and more material can be put on a roll without the risk of telescoping during processing. The resulting advantage is reduced machine downtime and operator intervention since the roll requires less frequent changes by comparison to the case in FIG. 5.

Although various embodiments have been illustrated, this was for the purpose of describing, but not limiting, the invention. Various modifications will become apparent to those skilled in the art and are within the scope of this invention, which is defined more particularly by the appended claims.

1. An absorbent article adapted to be worn in the crotch portion of a user's undergarment, said absorbent article having a transversely extending centerline and a longitudinally extending centerline substantially orthogonal to the transversely extending centerline, the article comprising:
a fluid-permeable layer which is oriented toward the user when said absorbent article is in use;
a liquid-impervious layer which is oriented toward the user's undergarment when said absorbent article is in use; and
a first liquid-absorbing component located between said fluid-permeable layer and said liquid-impervious layer, said first liquid-absorbing component comprising a first side and a second side generally opposite the first side, wherein the first side includes at least one projection and the second side includes at least one recess.

2. An absorbent article as defined in claim 1, wherein said first side and said second side extend along a transversely extending centerline.

3. An absorbent article as defined in claim 1, wherein said first side and said second side extend along a longitudinally extending centerline.

4. An absorbent article as defined in claim 2, wherein when said first side is subjected to an imaginary translation toward the second side along the longitudinally extending centerline, said projection being capable of at least partially penetrating said recess.

5. An absorbent article as defined in claim 4, wherein when said first side is subjected to an imaginary translation toward said second side along the longitudinally extending centerline, said projection being capable of matingly engaging said recess.

6. An absorbent article as defined in claim 1, wherein when said first side is subjected to an imaginary translation toward said second side along the longitudinally extending centerline, said first side includes a plurality of projections and said second side includes a plurality of recesses, wherein the said plurality of projections are capable of at least partially penetrating said recesses.

7. The absorbent article of claim 1 further comprising a second liquid absorbent component located between said fluid-permeable layer and said liquid-impervious layer.

8. The absorbent article of claim 7 wherein said second liquid-absorbing component comprise a third side and a fourth side generally opposite the third side, wherein the third side includes at least one projection and the fourth side includes at least one recess.

9. An absorbent article as defined in claim 1, wherein said liquid-absorbing component has a plurality of superposed layers including at least a first layer and a second layer, said first layer being attached to said second layer through at least one fold line, and wherein the first side of the liquid-absorbent component includes a plurality of first contours defined by said plurality of layers, and wherein the second side of the liquid-absorbent component includes a second plurality of contours defined by said plurality of layers, wherein at least one of the first contours includes said projection, and wherein at least one of the second contours includes said recess.

10. An absorbent article adapted to be worn in the crotch portion of a user's undergarment, said absorbent article comprising:
a fluid-permeable layer which is oriented toward the user when said absorbent article is in use;
a liquid-impervious layer which is oriented toward the user's undergarment when said absorbent article is in use;
at least one liquid-absorbing component located between said fluid-permeable layer and said liquid-impervious layer, said at least one liquid-absorbing component having a longitudinally extending centerline, a transversely extending centerline perpendicular to the longitudinally extending centerline, a first transversely extending side and a second transversely extending side generally opposite to the first transversely extending side, and wherein the first transversely extending side and the second transversely extending side are oriented along the transversely extending centerline, and wherein the first transversely extending side has a first contour that includes at least one projection and the second transversely extending side has a second contour that includes at least one recess, wherein the first contour and the second contour are capable of substantial coincident alignment when the first contour is subject to an imaginary translation along a longitudinally extending centerline of the liquid-absorbing component.

11. A method of making a liquid-absorbing component for an absorbent article, said method comprising:
providing a continuous web of liquid-absorbing material; and
separating a portion of the liquid-absorbing material from the continuous web to form a liquid-absorbing element, wherein said liquid-absorbing element includes a first side and a second side generally opposite the first side, and wherein the first side includes at least one projection and the second side includes at least one recess.

12. A method as defined in claim 11, wherein said continuous web is a roll of material and the method further comprises progressively unwinding the roll of material.

13. A method as defined in claim 11, wherein said separating is done at a die-cutting station comprising a roller with a circumferential face having at least one cutting blade.

14. A method as defined in claim 13, wherein said cutting blade is patterned according to the first side and the second side of said liquid-absorbing component.
